(19) United States
(12) Patent Application Publication
(10) Pub. No.: US 2019/0134064 A1
(43) Pub. Date: May 9, 2019

(54) SELECTIVELY DEGLYCOSYLATED VITAMIN D-BINDING PROTEIN (GCMAF), CHOLECALCIFEROL (CALCIOL), AND PRODUCTION METHOD

(71) Applicant: HG PHARMA GMBH, Wien (AT)

(72) Inventors: Ralf HERWIG, Westendorf (AT); Joachim GREILBERGER, Judendorf-Strassengel (AT)

(21) Appl. No.: 16/095,146

(22) PCT Filed: Apr. 21, 2017
(86) PCT No.: PCT/AT2017/060104

§ 371 Date: Oct. 19, 2018

(30) Foreign Application Priority Data
Apr. 21, 2016 (AT) A 50354/2016

(51) Int. Cl. A61K 31/593; A61K 38/38

(52) U.S. Cl. CPC A61K 31/593; A61P 35/00; A61K 38/38

(57) ABSTRACT

The invention relates to a dimeric complex of a selectively deglycosylated vitamin D-binding protein (GcMAF) and cholecalciferol (calciol). The invention further relates to a method for producing such a complex, wherein a vitamin D-binding protein is reacted with calciol and is selectively deglycosylated before or after being reacted. The resulting product is finally separated from impurities with a larger molecular weight, in particular enzymes of the selective deglycosylation process, using a molecular sieve with a cutoff value of more than 60 kDa.

### TECHNICAL FIELD

The present invention relates to selectively deglycosylated vitamin D-binding protein (GcMAF), cholecalciferol (calciol), and a production method.

### PRIOR ART

Cholecalciferol, also referred to as calciol, is known to be the most important physiological form of vitamin D in humans. For this reason, it is often used in dietary supplements. It is converted in the body to calcitriol. Calcitriol differs from calciol in that it has two OH groups at positions 1 and 25.

Calciol, as well as calcidiol and calcitriol, are hydrophobic; vitamin D is therefore transported in the blood by vitamin D-binding protein, which is also referred to as Gc-globulin (Gc=group specific component). When vitamin D-binding protein is selectively deglycosylated, one obtains a molecule that is an effective macrophage activator. This is known for example from paragraph [0009] of Patent Citation 0001: WO WO 01/85194 A. This molecule is therefore referred to in the following as GcMAF (MAF=macrophage-activating factor). It is known from the above-mentioned document that GcMAF is effective for example in treating tumors. It is thought that GcMAF activates macrophages, which then in turn attack tumor cells (paragraph [0010] of the aforementioned document).

On the other hand, calcitriol is also known to be helpful in treating tumors, particularly in prostate cancer (paragraph [0064] of said document).

In said document, therefore, it is also proposed to administer a composition comprising GcMAF and calcitriol to cancer patients (paragraph [0016]), particularly to patients with prostate cancer and breast cancer (paragraph [0066]). As no specific production method is indicated, it is to be assumed that the two compositions are present separately from one another and are not chemically bonded to each other.

According to Patent Citation 0002: WO WO 2014/202956 A, a trimeric complex of GcMAF, calcitriol, and an unsaturated fatty acid is produced. GcMAF is first reacted with calcitriol so that a dimeric complex, GcMAF-calcitriol, is produced as an intermediate product.

In the context of the present invention, it has now surprisingly been found that the complex GcMAF-calciol is superior to the complex GcMAF-calcitriol: its phagocytic activity is higher, and the formation of superoxide radical anions (O₂^{·-}) is lower.

### DESCRIPTION OF THE INVENTION

The invention therefore relates to a dimeric complex of a selectively deglycosylated vitamin D-binding protein (GcMAF) and cholecalciferol (calciol), particularly as a medicinal product.

As was further established in the context of the present invention, efficacy depends to a significant extent on the quality of purification. The invention therefore also relates to a method for producing such a complex, in which a vitamin D-binding protein is reacted with calciol and selectively deglycosylated before or after being reacted, wherein the resulting product is finally separated from impurities with a larger molecular weight, in particular enzymes of the selective deglycosylation process, using a molecular sieve with a cutoff value of more than 60 kDa, and preferably is also separated from cleaved sugar residues using a molecular sieve with a cutoff value of at most 10 kDa.

According to the measurements carried out in the context of the present invention, the phagocytic activity of the known complex GcMAF-calcitriol is virtually identical to the phagocytic activity of GcMAF alone; in contrast, the complex GcMAF-calciol has a significantly higher phagocytic activity.

Reduced production of superoxide radical anions is an advantage: although superoxide radical anions damage tumor cells, they also cause the same degree of damage to healthy cells and macrophages, which can then exert only a minor action in tumor treatment, and this more than negates the direct action of the superoxide radical anions.

The standard test for vitamin D in the blood, the ELISA test, is selective for calcidiol (which has an OH group at position 25). In this test, it is first necessary to separate the calcidiol from the vitamin D-binding protein, as the complex Gc-calcidiol does not bind to the receptor sites of the ELISA test. As the receptor sites of the ELISA test are similar to the vitamin D receptors in the human body, one can conclude from this that this complex also does not bind to these vitamin D receptors. The same applies for the complex GcMAF-calcitriol. Surprisingly, however, it has been found that the complex GcMAF-calciol does indeed bind to the receptor sites of the ELISA test, and thus also to the vitamin D receptors of the human body; this is probably the reason for its superior action.

### Embodiment(s) of the Invention

The invention will now be explained in further detail by means of the following description.

The production method will first be described: the complex according to the invention is produced from Gc-protein or recombinant Gc-protein with subsequent cholecalciferol binding.

Production of the immobilized enzyme by means of Sepharose 4B or cyano-activated magnetic beads or other cleavage possibilities

Buffers or solutions used:
Coupling buffer: dissolve 16.8 g of NaHCO₃ and 58.44 g of NaCl in 2 l of water
0.2 M glycine buffer: dissolve 15.01 g of glycine in 1 l of water
8 M NaOH concentrate: dissolve 32 g of NaOH in 100 ml of water
0.1 M acetate buffer: add 5.76 ml of glacial acetic acid (100%) to 1 l of water, dissolve 29.22 g (0.5 M) of NaCl therein, and add the 8 M NaOH concentrate dropwise to a pH of 4.0 (at least 50 drops)
Water: osmotically purified (also suitable for operations; conductivity<0.2 μS/cm)

A.) Cyano-activated Sepharose 4B:

1) Suspend 1.2 g of cyano-activated Sepharose 4B in 240 ml of 1 M HCl (cold; 83.3 g in 1 l) for 30 min. Transfer this to a chromatography column and flush with 60 ml of water. After this, equilibrate 60 ml of coupling buffer via the chromatography column. After rebuffering, the gel is transferred in 15 ml of coupling buffer into two 7.5 ml tubes and centrifuged for 2 min at 3000 rpm.

The supernatant is removed to 4.5 ml.

2.) The gels are now activated for the respective binding of:

1 ml of coupling buffer with 2 mg of galactosidase or 1 ml of coupling buffer with 5 U of neuraminidase

Add one substance each to the two 4.5 ml gels and swirl for 2 h at 26° C.

3.) Blocking of the residual activated CN groups of the Sepharose with glycine:

The two gels with the enzymes are transferred to a chromatography column and freed of unbound enzymes using 5×5 ml of coupling buffer. (Also possible by means of centrifugation: 6000 rpm, removal of the buffer from the gel, washing with coupling buffer, and repeated centrifugation; total of 5 times)

Finally:

Transfer in 2×5 ml of coupling buffer to 15 ml tubes and centrifuge for 2 min at 3000 rpm.

Then add 10 ml each of 0.2 M glycine, pH 8.0, and allow to stand for 20 h at 4-6° C.; swirl several times.

4.) Purification of the enzyme-activated gel of unbound glycine:

The tubes are centrifuged and the supernatant is removed.

Purification of the enzyme gel is carried out by 5-time addition of coupling buffer, centrifugation at 3000 rpm for 2 min, and removal of the supernatant.

After this, repeat the same procedure with 5-time addition of the acetate buffer instead of the coupling buffer.

Storage is carried out after washing 5 times with 5 ml of 1 M NaCl solution to which 0.05% sodium azide has been added.

Finally, the gel is transferred to a sterile tube and stored at 4-6° C. (=ready to use)

B.) Cyano-activated magnetic beads (CN-MB):

1.) Wash 1.2 g of CN-MB several times in 100 ml of 1 M HCl (cold; 83.3 g in 1 l) (by means of centrifugation and magnetic binding). After this, wash with water and equilibrate with coupling buffer.

After rebuffering, the 1.2 g of CN-MB is transferred to two 0.5 ml tubes and centrifuged for 2 min at 3000 rpm. The supernatant is removed.

2.) The magnetic beads are now activated for the respective binding of:

1 ml of coupling buffer with 2 mg of galactosidase

1 ml of coupling buffer with 5 U of neuraminidase

Add one substance each to 0.6 g of CN-MB and swirl for 2 h at 26° C.

3.) Blocking of the remaining activated CN groups of the magnetic beads with glycine:

Both CN-MB enzymes are held back by means of magnets, and the supernatant is removed. Addition of 1 ml of coupling buffer, shaking, and again magnetic binding of the CN-MG enzyme and removal of the supernatant. Repeat 4 times. This removes unbound enzymes.

Then add 1 ml each of 0.2 M glycine, pH 8.0, and allow to stand for 20 h at 4-6° C.; swirl several times.

4.) Purification of the enzyme-activated magnetic beads of unbound glycine:

Principle: magnetic binding and removal of purification buffer

5-time addition of 1 ml each of coupling buffer; after this, repeat the same procedure with 5-time addition of 1 ml of the acetate buffer.

Storage of galactosidase- and sialidase-MBs is carried out after washing five times with 1 ml of 1 M NaCl solution to which 0.05% sodium azide has been added in 0.5 ml of such a solution at 4-6° C. (=ready to use).

Cleavage of galactose or sialic acid (neuraminic acid) with CN-Sepharose 4B activated by means of galactosidase and sialidase (or neuraminidase) or CN-activated magnetic beads.

Buffers used:

0.1 M phosphate buffers, pH 6.0 and 7.0

1 mg of Gc-proteins is dissolved in 1 ml of 0.1 M phosphate buffer, pH 6.0.

400 μg thereof is dialyzed in 250 ml of water at room temperature (dialysis hose; filtration tube); Duration: 24 h, changed 3×.

A.) Neuraminidase cleavage:

The purified Gc-protein is brought into contact in 10 ml tubes with the immobilized neuraminidase (sialidase), as produced above, either with 3 ml of activated sepharose or with 0.5 ml of activated magnetic beads, and incubated at 37° C., 500 rpm, for 2 h.

After incubation

a.) For neuraminidase-activated Sepharose gel: centrifuge at 6000 rpm for 2 min, then remove 900 μl.

Mix the sialidase gel with 1 ml of 0.1 M phosphate buffer, pH 7.0 (=re-wash once), centrifuge again for 2 min at 6000 rpm, and again remove 900 μl. Re-wash two more times, centrifuge, and remove. Combine the removed supernatants.

b.) For neuraminidase-activated magnetic beads: hold back the magnetic beads and remove 900 μl.

Add 1 ml of 0.1 M phosphate buffer, pH 7.0, to the MB, mix, and again remove 900 μl. Carry out procedure a third time, and purify the removed supernatants.

Purification steps for cleavage products of a) and b): centrifuge via a filter (10,000 Da filter) at 6000 rpm for 5 min in order to remove the cleaved sugar residues.

Take up the supernatant in 4 ml of 0.1 M phosphate buffer, pH 7.0, and filter again. Carry this out a third time. Take up the supernatant in 1 ml of 0.1 M phosphate buffer, pH 7.0.

B.) Cleavage with galactosidase takes place similarly to cleavage with sialidase or neuraminidase via activated Sepharose or magnetic beads.

Isolation of the cleaved products, rebuffering, purification

a) For Sepharose:

Suspend the filter residue with 1 ml of 0.9% NaCl solution. After this, centrifuge for 2 min at 6000 rpm and remove 1 ml.

Suspend remainder with 1 ml of 0.9% NaCl solution, centrifuge again for 2 min at 6000 rpm, and remove 1 ml. Repeat this twice.

b) For magnetic beads:

Suspend the filter residue with 1 ml of 0.9% NaCl solution. After this, simply hold the beads back with a magnet and remove the protein (=α-globulin-N-acetylglucosamine). After this, add 1 ml each of 0.9% NaCl solution, mix, apply the magnet, and remove the supernatant. Repeat procedure 3 times.

Centrifuge all fractions together via 10,000 Da filter for 8 min at 6000 rpm to concentrate the solution. (Alternatively, pressure dialysis can be carried out.)

Add 4 ml of 0.9% NaCl solution to the residue and filter again for 10 min at 6000 rpm. The residue is approximately 500 μl.

Again add 4 ml of 0.9% NaCl solution, and filter again for 10 min at 6000 rpm; take up the residue in 1 ml of 0.9% NaCl solution.

Result: sialic acid separated; galactose separated; no DNA, RNA.

Finally, filtering is carried out via a 100,000 Da filter by means of centrifugation. GcMAF is located in the filtrate (about 53 kDa), possible enzymes of the immobilized material are separated in the filter residue. This yields extremely high purity of the product of over 99.5%.

After this, cholecalciferol is added in order to form the complex GcMAF-cholecalciferol. The resulting complex is separated from excess cholecalciferol using a 10,000 Da molecular filter (centrifuge 3×10 min at 6000 rpm and wash with 10 mM PBS, pH 7.4). Protein determination showed 398 ng/ml of the desired complex.

However, it is also possible to initially react cholecalciferol with the Gc-protein and then cleave the sugar residues as described above.

For the subsequent comparative tests, the same production method was carried out with calcitriol instead of cholecalciferol. The yield was 440 ng/ml of the complex.

Phagocytosis activation was determined as described by Hammarstrom S and Kabat EA in “Studies on specificity and binding properties of the blood group A reactive hemagglutinin from Helix pomatia”, Biochemistry 10: 1684-1692, 1971.

Peritoneal mouse cells were coated in a 24-well plate onto cover glasses. After three hours of drug treatment, the cultures were tested for phagocytic activity. Sheep red blood cells (SRBC) were opsonized using hemolytic rabbit serum (anti-sheep red blood C12HSB cells, Serotec Ltd. England). Opsonized SRBC (0.5%) in RPMI 1640 (serum-free) were layered onto each macrophage-coated cover glass and cultured for 90 minutes. The non-internalized erythrocytes were lyzed by immersing the cover glass in a hypotonic solution (⅕ PBS). The macrophages were fixed with methanol, air-dried, and Giemsa stained. The number of phagocytosed erythrocytes per cell was microscopically determined; 250 macrophages were counted for each data point. The data are expressed as a phagocytosis index, which is defined as the percentage of macrophages with engulfed erythrocytes multiplied by the average number of erythrocytes per macrophage.

The result is as follows:

TABLE 1

| Substance | Macrophage activity |
|---|---|
| GcMAF-calciol | 83.5 |
| GcMAF-calcitriol | 73.2 |
| GcMAF, produced as described | 73.0 |
| GcMAF, purchased from Metavectrum, Germany | 68 |

The higher activity of the self-produced GcMAF is thought to be attributable to the better purification, particularly the final filtration with the 100,000 Da filter.

Determination of the formation of superoxide radical anions in monocytes from human whole blood was carried out as follows:

Isolation of peripheral mononuclear cells is carried out by density gradient centrifugation from heparinized whole blood. The blood is diluted 1:2 with PBS (without Ca/Mg) and layered onto 15 ml of Histopaque-1077. After this, the cell suspension is centrifuged for 30 min at 700×g and 20° C. The interphase with the mononuclear cells is carefully removed with a Pasteur pipette, made up with 30 ml of PBS (without Ca/Mg), and centrifuged for 5 min at 350×g. The supernatant is decanted, and the cells are taken up in RPMI 1640 medium and counted in the flow cytometer. The cells are adjusted to 10⁵ monocytes/ml. For the test batch, 1 ml of cell suspension is pipetted into each 5 ml tube. LPS 100 ng/ml is used as a control. The test substance (GcMAF, GcMAF-calciol or GcMAF-calcitriol) is used in a concentration of 50 pg/ml. The cells are incubated for a total of 3 h in the CO₂ incubator. 15 min before the end of incubation, 5 μl each of CD45-V450 antibody and 1 μl of a 1 mM MitoSOX stock solution are added. After this, the cell suspension is made up with 2 ml each of RPMI medium and centrifuged for 5 min at 350×g at room temperature. The supernatant is decanted, and the cells are resuspended in 500 μl of RPMI medium. The cells are measured and analyzed in the flow cytometer. Evaluation is carried out based on the average fluorescence intensity, as described by Saharuddin Bin Mohamad, Hideko Nagasa Wa, Yoshihiro Uto, and Hitoshi Hori in “Preparation of Gc Protein-Derived Macrophage Activating Factor (GcMAF) and its Structural Characterization and Biological Activities”, Anticancer Research 22: 4297-4300 (2002).

Superoxide formation assay

The method used was a modified version of that described by Johnston et al. In brief, after three-hour treatment of the samples, the plates were washed twice with PBS (-) and once with Krebs-Ringer phosphate buffer, pH 7.4, 1.5 ml of 50 μM cytochrome C in Krebs-Ringer phosphate buffer was added, and phorbol myristate acetate (PMA) was added to a final concentration of 5 μg/ml in each well and cultured for 90 minutes in a moistened incubator.

The reaction was stopped using an ice bath. The cultured medium was placed in an Eppendorf tube and centrifuged at 8000 g. The optical density of the supernatant was spectrometrically determined at 550 nm with reference at 540 nm (U-2000, Hitachi) using mixtures of plates without cells as a blank measurement. The concentration of reduced cytochrome C was determined using the equation E₅₅₀ₙₘ=2.1·10⁻¹ M⁻¹cm⁻¹.

The result was as follows:

TABLE 2

| Substance | Superoxide activity |
|---|---|
| GcMAF-calciol | 142 |
| GcMAF-calcitriol | 188 |
| GcMAF, produced as described | 199 |
| GcMAF, purchased from Metavectrum, Germany | 150 |

It can be seen that the superoxide activity of the complex according to the invention is the lowest of all of the substances tested and is comparable to that of pure GcMAF. Binding of calciol to GcMAF can therefore significantly increase its macrophage activity, but without meaning that the disadvantage of an increase in superoxide activity must be accepted in order to achieve this increase.

The complex according to the invention can be orally administered. The vitamin D content in the blood was measured after oral administration, and it rose from 35.9 nM (before administration) to 44.25 nM (12 h after administration). The complex according to the invention is therefore absorbed by the body in oral administration. Moreover, the complex according to the invention is not toxic.

It was also possible to demonstrate a significant increase in monocytic cells, which were subsequently converted into macrophages, after oral administration of the complex according to the invention.

1. A dimeric complex of a selectively deglycosylated vitamin D-binding protein (GcMAF) and cholecalciferol (calciol).

2. The complex as claimed in claim 1 as a drug.

3. A method for producing a complex as claimed in claim 1, wherein a vitamin D-binding protein is reacted with calciol and selectively deglycosylated before or after being reacted, wherein the resulting product is finally separated from impurities with a larger molecular weight, in particular enzymes of the selective deglycosylation process, using a molecular sieve with a cutoff value of more than 60 kDa.

4. The method as claimed in claim 3, wherein the resulting product is separated from cleaved sugar residues using a molecular sieve with a cutoff value of at most 10 kDa.
